(19) United States
(12) Patent Application Publication
(10) Pub. No.: US 2021/0343412 A1
(43) Pub. Date: Nov. 4, 2021

(54) INTERVENTION CONTENT ESTIMATION DEVICE, METHOD, AND PROGRAM

(71) Applicant: NIPPON TELEGRAPH AND TELEPHONE CORPORATION, Tokyo (JP)

(72) Inventors: Hisashi KURASAWA, Musashino-shi, Tokyo (JP); Shozo AZUMA, Musashino-shi, Tokyo (JP); Naoki ASANOMA, Musashino-shi, Tokyo (JP); Akihiro CHIBA, Musashino-shi, Tokyo (JP); Kana EGUCHI, Musashino-shi, Tokyo (JP); Tsutomu YABUUCHI, Musashino-shi, Tokyo (JP); Kazuhiro YOSHIDA, Musashino-shi, Tokyo (JP); Tomohiro YAMADA, Musashino-shi, Tokyo (JP)

(73) Assignee: NIPPON TELEGRAPH AND TELEPHONE CORPORATION, Tokyo (JP)

(21) Appl. No.: 17/271,295

(22) PCT Filed: Jul. 16, 2019
(86) PCT No.: PCT/JP2019/027913

§ 371 Date: Feb. 25, 2021

(30) Foreign Application Priority Data
Aug. 31, 2018 (JP) 2018-163519

(51) Int. Cl. G16H 50/20; G16H 10/60; G06N 20/00

(52) U.S. Cl. CPC G16H 50/20; G06N 20/00; G16H 10/60

(57) ABSTRACT

An object of an aspect of the present invention is to enable estimation of more effective intervention content in order to make a person's health state approximate to an ideal health state, and in a learning phase, measurement values and target values of a health state for a plurality of days in the past are sequentially input to a learning machine configured by a multilayer neural network, and the learning machine is caused to perform learning such that a target achievement expectation value obtained by using a success rate that allows the user's health state to approximate to an ideal health state, continuity that allows the health state approximate to the ideal health state to be maintained, and a target value of the health state to be subsequently recommended and the target achievement expectation value thereof that reflect a temporal change in the health state and a history of interventions until a present time are output. In an estimation phase, measurement values and target values of the user's health state in the last three days are input to the estimation model after learning, and the target value of the health state to be recommended, which is output from the estimation model at this time, is presented to the user.

### TECHNICAL FIELD

The invention relates to an apparatus, a method, and a program adapted to estimate a target value of a health state for making a person's health state approximate to an ideal health state, for example.

### BACKGROUND ART

Lifestyle-related diseases are a group of disorders of which occurrence and progress are significantly affected by lifestyle habits such as diet, physical activity, sleep, and alcohol intake, and include diabetes, cancer, and the like. It is known that active intervention is effective for patients in pre-symptomatic states or in early stages of development of diseases to prevent the development and progress of the lifestyle-related diseases. Examples of intervention in diet include limitation of intake calories, designation of order of eating, and limitation of eating times. Examples of intervention in physical activity include designation of amounts of exercise, designation of exercise times, and designation of exercise types such as swimming and jogging. Examples of intervention in sleep include designation of lengths of sleeping times and bedtimes and wakeup times. Examples of intervention in alcohol intake include limitation of the amounts of alcohol intake and alcohol intake intervals.

Thus, an effort of uniquely setting a target value derived from an ideal health state and presenting the target value as intervention content has been proposed in the related art. For an intervention in physical activity, for example, a uniform target such as 10000 steps per day is presented to promote a change in action. For treatment after the development of a lifestyle-related disease, HbA1c (NGSP) 7% is presented as a blood glucose management target value for diabetes treatment to enhance adherence to the treatment (see Non Patent Literature 1, for example).

### CITATION LIST

### Non Patent Literature

Non Patent Literature 1: The Japan Diabetes Society, Kumamoto Declaration 2013, —For You and Your Important Persons, Keep your A1C below 7%—, 2013, Internet <URL: http://www.jds.or.jp/common/fckeditor/editor/filemanager/connectors/php/transfer.php?file=/uid000025_6B756D616D6F746F323031332E706466>

### SUMMARY OF THE INVENTION

### Technical Problem

However, an intervention method proposed in the related art is adapted to uniquely set a target value derived simply from an ideal health state. Thus, because a current health state of a person is not taken into consideration, an effect thereof for adherence representing a change in action and a status of compliance with the intervention is limited.

The present invention was made in view of the aforementioned circumstances, and an object thereof is to provide a technique that enables estimation of a more effective target value of a health state as intervention content in order to make a person's health state approximate to an ideal health state.

### Means for Solving the Problem

In order to achieve the aforementioned object, a first aspect of the present invention provides an intervention content estimation apparatus including: a first acquisition portion configured to acquire, on a per user basis, record information that includes a target value of a health state determined based on a current health state and a future ideal health state set in advance, and a measurement value of the health state of the user after a target value of the health state to be subsequently recommended is presented; and an estimation model learning portion configured to generate an intervention content estimation model by inputting the record information acquired by the first acquisition portion as training data to a learning machine and causing the learning machine to perform learning such that a target value of the health state to be subsequently recommended is output as an evaluation result from the learning machine.

A second aspect of the present invention further includes: a second acquisition portion configured to acquire, on a per user basis, most recent record information including the target value of the health state presented and the measurement value of the health state after the target value of the health state is presented; and an intervention content estimation portion configured to input the most recent record information acquired by the second acquisition portion as evaluation data to the intervention content estimation model and output, as estimation data, information representing a target value of the health state to be subsequently recommended, which is output from the intervention content estimation model in response to the input.

### Effects of the Invention

According to the first aspect of the present invention, the record information including a target value of a health state determined based on a current health state of the user and a future ideal health state set in advance, and a measurement value of the health state of the user after the target value is presented is input as training data, and an intervention content estimation model after learning is generated that can output, as an evaluation result, information representing a target value of the health state to be subsequently recommended to the user. It is thus possible to provide an intervention content estimation model capable of estimating a target value of a more effective health state in order to make the user's health state approximate to an ideal health state rather than uniquely presenting a uniform target value.

According to the second aspect of the present invention, the record information including the target value of the most recent health state and the measurement value of the health state after the target value is presented, is input as the evaluation data to the estimation model, and the information representing the target value of the health state that is to be recommended next is thereby output as the estimation data in response to content of the input. It is thus possible to present, to the user, a more effective target value of the health state in order to make the user's health state approximate to the ideal health state and thereby to expect a higher effect for adherence to a change in action and intervention as compared with a case in which a uniform target value is uniquely presented.

### BRIEF DESCRIPTION OF DRAWINGS

FIG. 1 is a block diagram illustrating a functional configuration of an intervention content estimation apparatus according to an embodiment of the present invention.

FIG. 2 is a flowchart illustrating a processing procedure and processing content of a learning phase performed by the intervention content estimation apparatus illustrated in FIG. 1.

FIG. 3 is a flowchart illustrating a processing procedure and processing content of an estimation phase performed by the intervention content estimation apparatus illustrated in FIG. 1.

FIG. 4 is a diagram illustrating an example of training data used in a learning phase illustrated in FIG. 2.

FIG. 5 is a diagram illustrating an example of a configuration of an intervention content estimation model.

FIG. 6 is a diagram illustrating an example of an estimation result obtained by an intervention content estimation apparatus according to an embodiment of the present invention.

FIG. 7 is a diagram illustrating an example of a case in which a target value of a health state is uniformly set in the related art.

### DESCRIPTION OF EMBODIMENTS

Hereinafter, an embodiment of the present disclosure will be described in detail with reference to the drawings.

### Embodiment

### Configuration Example

According to an embodiment of the present invention, an intervention content estimation model is generated through deep reinforcement learning in order to make a user's health state approximate to a future ideal health state. Here, the intervention content estimation model uses, as inputs, measurement values of a health state on a plurality of days in the past and target values of the health state presented to the user in the plurality of days. The intervention content estimation model outputs a target value of a health state to be subsequently recommended to the user and a target achievement expectation value thereof. Thereafter, the intervention content estimation model is caused to output the target value of the health state to be subsequently recommended and the target achievement expectation value thereof, and present the target value and the target achievement expectation value as intervention content to the user.

Note that although the number of steps and the intake calories are considered as parameters indicating a health state, the parameters are not limited thereto and may be lifestyle habits such as diet, physical activity, sleep, and alcohol intake, and values of sample tests and physiological tests. As intervention content, it is possible to apply content related to lifestyle habits such as diet, physical activity, sleep, and alcohol intake and the values of sample tests and physiological tests in addition to the number of steps and the intake calories.

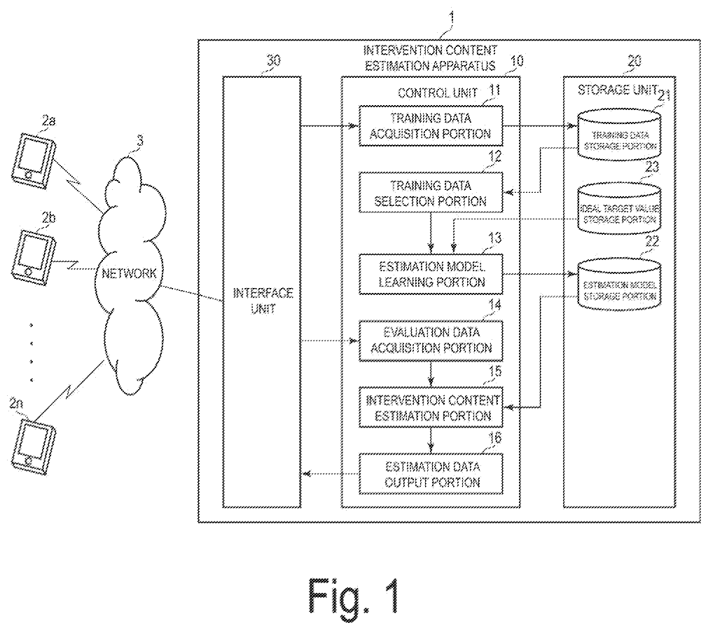

FIG. 1 is a block diagram illustrating a functional configuration of the intervention content estimation apparatus according to an embodiment of the present invention.

The intervention content estimation apparatus 1 is configured by, for example, a server computer or a personal computer, and can communicate with a plurality of user terminals 2a to 2n via a network 3.

The user terminals 2a to 2n are owned by different users and include, for example, smartphones, tablet-type terminals, or personal computers. The user terminals 2a to 2n have a pedometer or a function of measuring intake calories in the user terminals themselves or have a function of receiving the numbers of steps and the intake calories measured by external measurement equipment through communication mechanisms or manual inputs and storing the numbers of steps and the intake calories as information representing users' health states.

Also, the user terminals 2a to 2n have a function of receiving target values of health states to be recommended, which are transmitted from the intervention content estimation apparatus 1, and displaying the target values to the users. Further, the user terminals 2a to 2n generate time-series data that associates measurement values representing health states and the target values of the recommended health states with date information of each day, for example, and store the time-series data as record information. The user terminals 2a to 2n have a function of reading the time-series data in response to users' transmission operations or a transmission request from the intervention content estimation apparatus 1 and transmitting the time-series data to the intervention content estimation apparatus 1.

Each of the aforementioned functions that the user terminals 2a to 2n have is implemented by an application program installed in advance. Note that, as the user terminals 2a to 2n, wearable terminals each including a pedometer, a function of measuring intake calories, and a communication function can be used.

The network 3 includes, for example, a public network such as the Internet and an access network for accessing the public network. A local area network (LAN) or a wireless LAN, for example, is used as the access network, and a wired telephone network, a cable television (CATV) network, a mobile phone network, or the like can be used.

The intervention content estimation apparatus 1 is operated by, for example, a medical institution, a health support center, a fitness club, or other health support service operators and is configured by, for example, a server computer or a personal computer. Note that a stand-alone intervention content estimation apparatus 1 may be installed. The intervention content estimation apparatus 1 may be provided, as an expanded function, in a terminal of a clinician such as a doctor, an electronic medical record (EMR) server placed in an individual medical institution, an electronic health record (EHR) server placed in an individual area including a plurality of medical institutions, a cloud server of a service operator, or the like. Further, the intervention content estimation apparatus 1 may be provided as an expanded function in each of the user terminals 2a to 2n themselves.

The intervention content estimation apparatus 1 includes a control unit 10, a storage unit 20, and an interface unit 30. The interface unit 30 performs data transmission between itself and the user terminals 2a to 2n via the network 3. The interface unit 30 may have a function of performing data transmission with a management terminal (not illustrated) connected via a LAN or a signal cable.

The storage unit 20 is configured by combining, as a storage medium, a nonvolatile memory in which writing and reading can be performed any time, such as a hard disk drive (HDD) or a solid state drive (SSD), a nonvolatile memory such as a read only memory (ROM), and a volatile memory such as a random access memory (RAM), for example. In the storage regions thereof, a program storage region and a data storage region are configured. A program necessary to execute various kinds of control processing according to the embodiment of the present invention is stored in the program storage region.

In the data storage region, a training data storage portion 21, an estimation model storage portion 22, and an ideal target value storage portion 23 are configured. The training data storage portion 21 is used to store, as training data, time-series data of a plurality of days acquired from the user terminals 2a to 2n in the learning phase. The estimation model storage portion 22 is used to store the intervention content estimation model after learning. The ideal target value storage portion 23 stores ideal target values in advance.

The control unit 10 includes a hardware processor such as a central processing unit (CPU), for example, and has, as control functions for realizing the embodiment of the present invention, a training data acquisition portion 11, a training data selection portion 12, an estimation model learning portion 13, an evaluation data acquisition portion 14, an intervention content estimation portion 15, and an estimation data output portion 16. All of these control functional portions are implemented by causing the hardware processor to execute the program stored in the program storage region.

The training data acquisition portion 11 performs processing of acquiring, as training data, time-series data of a plurality of days in the past for each user from each of the user terminals 2a to 2n via the network 3 and the interface unit 30 and causing the training data storage portion 21 to store the acquired training data in association with individual identification information (a user ID) of each user, in a learning phase.

The training data selection portion 12 performs processing of sequentially selecting training data of a plurality of days stored in the training data storage portion 21 in units of three days while shifting the dates day by day, for example, and providing the training data to the estimation model learning portion 13.

The estimation model learning portion 13 uses deep reinforcement learning, for example, to cause the learning machine to perform learning such that a target value of a health state to be subsequently recommended and a target achievement expectation value thereof are output as estimation data of intervention content when the training data is input for each user. Here, the training data includes measurement values representing the health state in the past three days and the target value of the recommended health state. For the target value of the health state to be subsequently recommended and the target achievement expectation value, a probability at which a final target (ideal target value) can be achieved, continuity, a temporal change in health state and history of interventions until a present time are taken into consideration. Here, the probability at which the final target can be achieved is a target achievement expectation value obtained from a success rate at which the current health state can approximate to the target value corresponding to the ideal health state stored in the ideal target value storage portion 23. Also, the continuity is continuity that allows the health state approximate to the ideal health state to be maintained. The estimation model learning portion 13 causes the estimation model storage portion 22 to store the intervention content estimation model after learning. As the learning machine, a multilayer neural network, for example, is used. Note that a specific example of learning processing performed by the estimation model learning portion 13 will be described later.

The evaluation data acquisition portion 14 performs processing of acquiring time-series data that includes the measurement values representing the health state in the last three days, for example, and the target value indicating the health state recommended in the same period of time, and are transmitted from each of the user terminals 2a to 2n, via the network 3 and the interface unit 30 in response to an intervention content estimation request from each of the user terminals 2a to 2n, in the estimation phase.

The intervention content estimation portion 15 inputs the time-series data of the last three days acquired by the evaluation data acquisition portion 14 to the intervention content estimation model after learning that is stored in the estimation model storage portion 22. The intervention content estimation portion 15 performs processing of delivering to the estimation data output portion 16 a target value of a health state, output from the intervention content estimation model at this time, that is recommended to be used on the next day, as estimation data of intervention content. Note that the intervention content estimation portion 15 may save the estimation data of the intervention content in the estimation data storage portion (not illustrated) in the storage unit 20 in association with the date of the next day and the user ID.

The estimation data output portion 16 performs processing of generating estimation result notification data including the target value of the recommended health state, which has been delivered from the intervention content estimation portion 15, and transmitting the estimation result notification data from the interface unit 30 to one of the user terminals 2a to 2n originating a request.

### Operation Examples

Next, operation examples of the intervention content estimation apparatus 1 configured as described above will be described.

### (1) Learning Phase

Once the learning phase is set, the intervention content estimation apparatus 1 executes learning processing for the intervention content estimation model as follows.

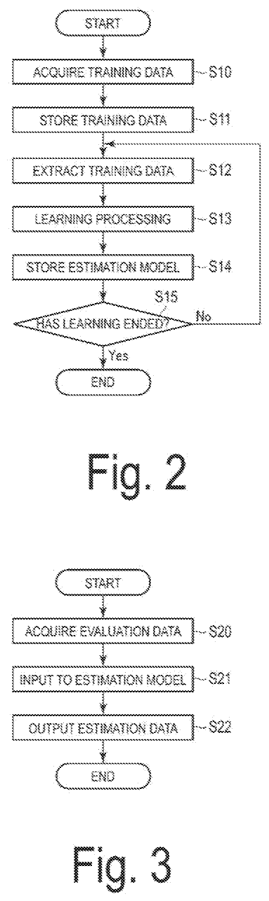

FIG. 2 is a flow diagram illustrating an example of a processing procedure and processing content in the learning phase performed by the control unit 10 of the intervention content estimation apparatus 1.

(1-1) Acquisition of Training Data

In each of the user terminals 2a to 2n, a target value of a recommended health state transmitted from the intervention content estimation apparatus 1 every day is displayed on a display portion and is stored in a storage portion in association with date information. Additionally, the number of steps measured by a pedometer and the intake calories manually input by the user, for example, are stored in the storage portion every day in association with the date information. In this manner, time-series data is sequentially stored on each day in the storage portion, the time-series data including measurement values of the number of steps and the intake calories representing the health state of that day, and the number of steps and the target value of intake calories representing the recommended health state transmitted from the intervention content estimation apparatus 1. The time-series data stored on each day is training data to be used by the intervention content estimation apparatus 1 to learn the estimation model.

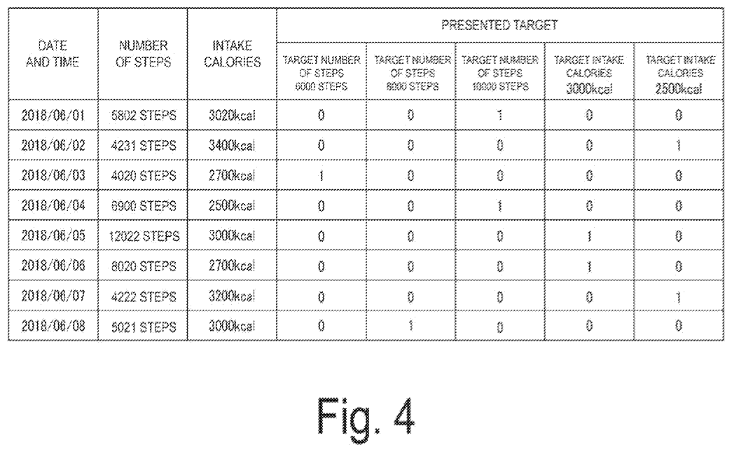

FIG. 4 illustrates an example of time-series data (training data) stored in the storage portion of each of the user terminals 2a to 2n. In this example, measurement values of the number of steps and the intake calories representing the daily health state in the period from Jun. 1 to Jun. 8, 2018 are stored. In this example, information designating any of “the target number of steps: 6000 steps”, “the target number of steps: 8000 steps”, “the target number of steps: 10000 steps”, “the target intake calories: 3000 kcal”, and “the target intake calories: 2500 kcal” is stored as the target value representing the recommended health state presented by the intervention content estimation apparatus 1. Here, an example in which a flag “1” is stored for the presented target while a flag “0” is stored for the other cases is illustrated.

The control unit 10 first accesses each of the user terminals 2a to 2n via the interface unit 30 under control of the training data acquisition portion 11 and thereby receives time-series data of eight days, for example, in Step S10. The time-series data is then stored in the training data storage portion 21 in association with each user ID in Step S11.

Note that in a case that the estimation data storage portion (not illustrated) is provided in the storage unit 20 of the intervention content estimation apparatus 1, the intervention content estimation apparatus 1 acquires only the everyday measurement values of the number of steps and the intake calories from each of the user terminals 2a to 2n. Also, the acquired measurement values of the number of steps and the intake calories, and flag information that represents the target value of the daily health state recommended for each user and is stored in the estimation data storage portion, may be associated with date, and training data may thus be acquired. Further, any number of days of time-series data may be acquired as long as the time-series data includes data of a plurality of days.

(1-2) Selection of Training Data

Once the time-series data of a plurality of days is acquired for each user, the control unit 10 of the intervention content estimation apparatus 1 reads the time-series data in units of three days from the training data storage portion 21 while shifting the date day by day, for example, under control of the training data selection portion 12 in Step S12. Then, the control unit 10 of the intervention content estimation apparatus 1 provides the time-series data of the three days as training data to the estimation model learning portion 13 under control of the training data selection portion 12.

If time-series data of eight days from Jun. 1 to Jun. 8, 2018 illustrated in FIG. 4 has been acquired and stored in the training data storage portion 21, for example, time-series data of three days from Jun. 1 to Jun. 3, 2018 is selected from the time-series data of the eight days first. The time-series data is selected as training data while sequentially shifting the date day by day such that the time-series data of three days from Jun. 2 to Jun. 4, 2018 is subsequently selected and time-series data of three days from Jun. 3 to Jun. 5, 2018 is then selected.

Note that although a case in which training data is selected in units of three days in learning processing performed once will be described here as an example, the training data may be selected in units of four or more days or in units of two days.

(1-3) Learning of Estimation Model

The control unit 10 of the intervention content estimation apparatus 1 then executes processing of causing the intervention content estimation model to perform learning as follows in Step S13 under control of the estimation model learning portion 13.

In other words, the estimation model learning portion 13 generates the intervention content estimation model through deep reinforcement learning, for example. Appropriate intervention content, that is, a target value of a health state can be estimated on the basis of the target achievement expectation value through the deep reinforcement learning. Continuity of the intervention effect can be reflected by setting a parameter called a discount rate. A past intervention history can be reflected by allowing a plurality of days of data to be input at once as training data.

Through the deep reinforcement learning, both an agent and an environment are designed, for example. The agent selects what action is to be selected on the basis of an observed state, and the environment updates a state depending on the action. Then, a reward, that is, a success rate is determined on the basis of the updated state. In the embodiment, the agent corresponds to the intervention content estimation apparatus 1 and determines the target number of steps of the next day on the basis of the health state of each user in the last three days. As for the reward, clipping is introduced to promote the learning, and if the current health state satisfies not less than 10000 steps per day and the intake calories of less than 2500 kcal, which are set as the future ideal health state, the reward is set to +1, otherwise the reward is set to −1. The environment corresponds to each user and a measurement value of the number of steps on a day on which the target number of steps is presented is registered therein.

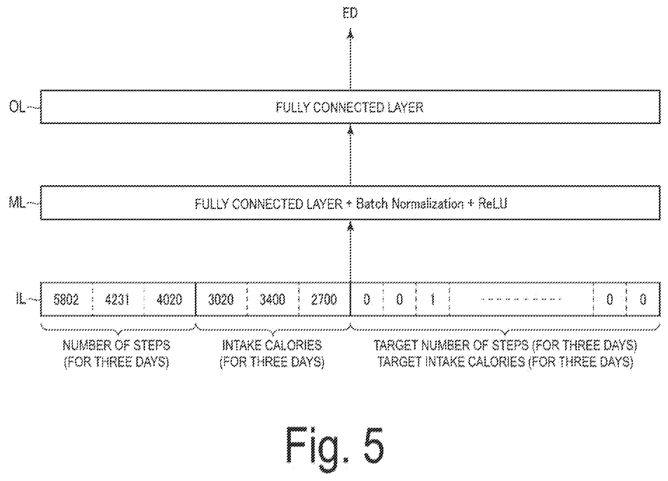

A Q function is constructed by a multilayer neural network. The multilayer neural network includes three fully connected layer as illustrated in FIG. 5, for example. Among the three layers, an input layer IL and an intermediate layer ML include a fully connected layer, Batch Normalization, and an activation function ReLU, and an output layer OL includes a fully connected layer.

A six-dimensional vector is constituted by measurement values of the number of steps and the intake calories in three days. Flag values (“1” or “0”) set for five target values of one day are connected to configure a fifteen-dimensional vector, the five target values being the target number of steps that is 6000 steps, the target number of steps that is 8000 steps, the target number of steps that is 10000 steps, the target intake calories that are 3000 kcal, and the target intake calories that are 2500 kcal of three days. Then, the six-dimensional vector of the measurement values of the health state and the fifteen-dimensional vector of the target values of the health state are connected to configure twenty one-dimensional vector, and the twenty one-dimensional vector is used as an input value for the input layer IL. In other words, the unit size of the input layer IL is “21”.

An output of the output layer OL is a five-dimensional vector representing the five target values and target achievement expectation values thereof. In other words, the unit size of the output layer is “5”. The unit size of the intermediate layer is configured to be “64”. Note that the parameters are not limited thereto, and the unit sizes can be changed in accordance with a reference period of time and the number of target options.

A discount rate of the reward (the parameter representing continuity) is configured to, for example, “0.9”. A correct answer of the Q function at a clock time t is defined as a value obtained by adding the reward (success rate) to a value obtained by multiplying a target achievement expectation value of the Q value by a discount rate as a coefficient. The estimation model learning portion 13 then learns the Q function such that a mean squared error of the right answer is minimized.

The estimation model learning portion 13 temporarily saves the parameters obtained by the learning processing in Step S14. Then, whether or not the learning processing on all the pieces of time-series data stored in the training data storage portion 21 has ended is determined in Step S15, and in a case in which unselected time-series data remains, the processing returns to Step S12, and the learning processing in Steps S12 to S14 is repeatedly executed. In contrast, once the learning processing on all the pieces of time-series data ends, the estimation model learning portion 13 causes the estimation model storage portion 22 to store the finally obtained parameters of the Q function as an intervention content estimation model and ends the processing.

(2) Estimation Phase

Once the estimation phase is set, the intervention content estimation apparatus 1 executes processing of estimating a target value of a recommended health state and a target achievement expectation value for each user as follows.

FIG. 3 is a flowchart illustrating an example of a procedure and processing content of intervention content estimation processing performed by the control unit 10 of the intervention content estimation apparatus 1.

(2-1) Acquisition of Evaluation Data

The user terminals 2a to 2n transmit time-series data of most resent three days of target users to the intervention content estimation apparatus 1. In response to this, in Step S20, the control unit 10 of the intervention content estimation apparatus 1 imports, as evaluation data, the time-series data of the last three days transmitted from the user terminals 2a to 2n via the interface unit 30 under control of the evaluation data acquisition portion 14. As illustrated in FIG. 4, for example, the time-series data includes measurement values of the number of steps and the intake calories representing a health state of the last three days of each user, and target values and target achievement expectation values of the number of steps and the intake calories presented by the intervention content estimation apparatus 1 in the past for the three days.

Note that an input of the measurement values of the number of steps and the intake calories to each of the user terminal 2a to 2n is performed by transferring each of the measurement values of a pedometer and a calorimeter to each of the user terminals 2a to 2n through communication or by inputting, by each user, each of the measurement values to each of the user terminals 2a to 2n in a manual operation.

(2-2) Estimation of Intervention Content

Once the import of the evaluation data ends, the control unit 10 of the intervention content estimation apparatus 1 then executes, under control of the intervention content estimation portion 15, processing of estimating the intervention content as follows.

In other words, the intervention content estimation portion 15 reads an estimation model after learning stored in the estimation model storage portion 22. Then, the acquired evaluation data is input to the input layer IL of the estimation model after learning as illustrated in FIG. 5 in Step S21. Here, the evaluation data is data of twenty one-dimensional vector including measurement values of the number of steps and the intake calories of the last three days, and target values of the number of steps and the intake calories presented by the intervention content estimation apparatus 1 in the past. Thus, arithmetic operations are performed by the input layer IL and the intermediate layer ML using the data of the twenty one-dimensional vector as an input in the estimation model after learning. Then, the target value and the target achievement expectation value of the number of steps and the intake calories to be recommended, which are represented by the five-dimensional vector, are output from the output layer as estimation data ED representing the intervention content of the next day in the estimation model after learning.

As a method for outputting the intervention content estimation data, the following two types of methods are conceivable, for example.

One of the methods is a method for selecting one of five options with the highest target achievement expectation value and configuring the selected one as estimation data ED, the five options being the target number of steps that is 6000 steps, the target number of steps that is 8000 steps, the target number of steps that is 10000 steps, the target intake calories that are 3000 kcal, and the target intake calories that are 2500 kcal.

The other method is a method for selecting N highest (for example, two highest) candidates of target value in a descending order from the highest target achievement expectation value from among the five options and configuring the selected ones as estimation data ED, the five options being the target number of steps that is 6000 steps, the target number of steps that is 8000 steps, the target number of steps that is 10000 steps, the target intake calories that are 3000 kcal, and the target intake calories that are 2500 kcal.

(2-3) Output of Estimation Data

In Step S22, under control of the estimation data output portion 16, the control unit 10 generates notification data that includes the estimated value indicating the intervention content of the next day and is output from the intervention content estimation portion 15, and transmits the notification data from the interface unit 30 to each of the user terminals 2a to 2n originating a request. Note that the transmission method may be a method for performing transmission from the intervention content estimation apparatus 1 in a form that allows the user terminal to view the notification data using a browser function or may be a method for performing transmission in the form of attachment to an email.

Once each of the user terminals 2a to 2n receives the notification data transmitted from the intervention content estimation apparatus 1, each of the user terminals 2a to 2n causes the display portion to display the information representing the target value of the number of steps or the intake calories that is recommended and included in the notification data, and stores the information as a component of time-series data in association with a corresponding date.

In a case that a plurality of N highest (for example, two highest) candidates of the target value of highest target achievement expectation values are included in the notification data at this time, the two candidates of the target value are each displayed to allow the user to select a preferred one. Each of the user terminals 2a to 2n stores the target value selected by the user as a component of time-series data in association with a corresponding date.

Effect

As described above in detail, according to the embodiment of the present invention, the measurement values and the target values of the health state in a plurality of days in the past are sequentially input to the learning machine configured by the multilayer neural network in units of three days, and the learning machine is caused to learn the values in the learning phase. At this time, the learning machine performs learning such that the target value of the health state to be subsequently recommended and the target achievement expectation value thereof are output.

Here, a target achievement expectation value obtained from a success rate that allows the user's health state to approximate to an ideal health state, continuity that allows the health state approximate to the ideal health state to be maintained, and a temporal change in health state and history of interventions until a present time are reflected in the target value of the health state to be subsequently recommended and the target achievement expectation value. Then, the measurement values and the target values of the user's health state in the last three days are input to the estimation model after learning in the estimation phase. Then, the target values of the health state to be recommended, which are output from the estimation model at this time, are transmitted as intervention content estimation data to the corresponding one of user terminals 2a to 2n and are presented to the user.

Thus, when target values of a health state are presented to a user, the subsequent target values of the health state are output on the basis of measurement values of the users health state in the most recent dates and target values of the health state that corresponds to the dates and are presented in advance. Here, a success rate that allows the health state to approximate to an ideal health state, continuity that allows the health state approximate to the ideal health state to be maintained, and a temporal change in health state and history of interventions until a present time are reflected on the subsequent target values of the health state. Thus, a steady effect is expected for achievement of the ideal health state, and effective intervention content for maintaining a state approximates to the ideal health state can be presented. Further, the intervention content in the past three days is taken into consideration, and it is possible to present highly effective intervention content in consideration of influences on a daily target value and a target achievement expectation value.

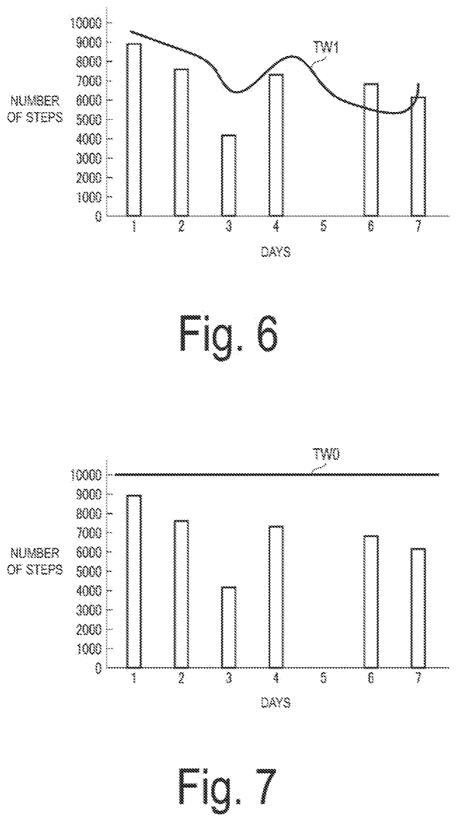

FIG. 6 illustrates an example of a change in target value TW1 of the number of steps presented on a daily basis as one item of intervention content according to an embodiment of the present invention. In contrast, FIG. 7 illustrates an example in the related art in which the target value TW0 of the number of steps is uniformly configured. According to the embodiment, it is possible to enhance an effect of adherence to a change in action or interventions by adaptively setting a target value of the number of steps on the next day in accordance with the most recent intervention content and a change in the number of steps after the intervention for the user rather than uniformly configuring the target value of the number of steps.

As a result, according to the embodiment, it is possible to expect a steady effect for the achievement of an ideal health state and present intervention content that contributes to an improvement in lifestyle, thus preventing regaining of lost weight caused due to fast weight loss, for example, and further allowing the intervention content to be presented that reduces a feeling of discomfort for the user in consideration of a correlation of intervention content.

Further, it is possible for the user to execute an action to make the health state approximate to its ideal through selective presentation of intervention content with the highest target achievement expectation value. On the other hand, it is also possible to allow the user to select desired intervention content by selecting an output method for presenting to the user a plurality of intervention content items with higher target achievement expectation values.

### Other Embodiments

The disclosure is not limited to the above-described embodiment. According to the embodiment, the case in which the functions of the intervention content estimation apparatus are provided on the server in the network has been described, and the functions may be provided in a user terminal as a part of expanded functions thereof, for example. This has an advantage that allows communication traffic and communication cost to be reduced though the user terminal has a higher processing load.

In addition, the functional configuration, the procedure and the processing content of the learning processing and the estimation apparatus, the types of information representing health states, and the like of the intervention estimation apparatus can also be implemented in variously modified manners without departing from the gist of the present invention.

The present invention is not limited to the embodiments described above, but various changes and modifications can be made without departing from the gist of the present invention. Furthermore, the embodiments may be implemented in combination appropriately as long as it is possible, and in this case, combined effects can be obtained. Further, the above embodiments include inventions on various stages, and various inventions may be extracted by appropriate combinations of the disclosed multiple configuration requirements.

### APPENDIX

Although some or all of the embodiments can also be described as in the following appendix in addition to the claims, the present invention is not limited thereto.

### APPENDIX 1

An intervention content estimation apparatus including:

a hardware processor,

in which the hardware processor executes the following two processes.

The first processing is processing of acquiring, for each user, record information including a target value of a health state determined on the basis of a current health state and a future ideal health state set in advance and a measurement value of a health state of the user after presenting the target value of the health state.

The second processing is processing of generating an intervention content estimation model by inputting the acquired record information as training data to a learning machine and causing the learning machine to perform learning such that information representing a target value of a health state to be subsequently recommended is output as an evaluation result from the learning machine.

### APPENDIX 2

An intervention content estimation apparatus configured such that the hardware processor further executes the following two processes.

The first processing is processing of acquiring, for each user, most recent record information including the presented target value of the health state and the measurement value of the user's health state after the target value of the health state is presented.

The second processing is intervention processing of inputting the most recent record information acquired by the second acquisition portion as evaluation data to the intervention content estimation model and outputting, as estimation data, information representing a target value of a health state to be subsequently recommended, which is output from the intervention content estimation model, in accordance with the input.

### APPENDIX 3

A storage medium storing a program that causes a hardware processor to execute the following two processes.

The first processing is processing of acquiring, for each user, record information including a target value of a health state determined on the basis of a current health state and a future ideal health state set in advance and a measurement value of a health state of the user after presenting the target value of the health state.

The second processing is processing of generating an intervention content estimation model by inputting the acquired record information as training data to a learning machine and causing the learning machine to perform learning such that information representing a target value of a health state to be subsequently recommended is output as an evaluation result from the learning machine.

### APPENDIX 4

A storage medium storing a program that causes the hardware processor to further execute the following two processes.

The first processing is processing of acquiring, for each user, most recent record information including the presented target value of the health state and the measurement value of the user's health state after the target value of the health state is presented.

The second processing is processing of inputting the most recent record information acquired by the second acquisition portion as evaluation data to the intervention content estimation model and outputting, as estimation data, information representing a target value of a health state to be subsequently recommended, which is output from the intervention content estimation model, in accordance with the input.

### REFERENCE SIGNS LIST

1 Intervention content estimation apparatus
2a to 2n User terminal
3 Network
10: Control unit
11 Training data acquisition portion
12 Training data selection portion
13 Estimation model learning portion
14 Evaluation data acquisition portion
15 Intervention content estimation portion
16 Estimation data output portion
20 Storage unit
21 Training data storage portion
22 Estimation model storage portion
23 Ideal target value storage portion
30 Interface unit

1. An intervention content estimation apparatus comprising:
a processor; and
a storage medium having computer program instructions stored thereon, when executed by the processor, perform to:
acquire, on a per user basis, record information that includes a target value of a health state, which is determined based on a current health state and a future ideal health state set in advance, and a measurement value of the health state of the user after the target value of the health state is presented; and
generate an intervention content estimation model by inputting the record information acquired by the first acquisition portion as training data to a learning machine and causing the learning machine to perform learning such that information representing a target value of the health state to be subsequently recommended is output as an evaluation result from the learning machine.

2. The intervention content estimation apparatus according to claim 1, wherein the computer program instructions further causes the learning machine to perform learning such that information reflecting a target achievement expectation value is output as the evaluation result, the target achievement expectation value being obtained by using a success rate that allows the current health state to approximate to the ideal health state.

3. The intervention content estimation apparatus according to claim 1, wherein the computer program instructions further
causes the learning machine to perform learning such that information reflecting a target achievement expectation value is output as the evaluation result, the target achievement expectation value being obtained by using a success rate that allows the current health state to approximate to the ideal health state, and a discount rate provided to the success rate as a coefficient.

4. The intervention content estimation apparatus according to claim 2, wherein the computer program instructions further perform to input the acquired record information of a plurality of days set in advance as training data to the learning machine and thereby causes the learning machine to perform learning such that information reflecting the target achievement expectation value, and a temporal change in the measurement value of the health state and a history of a change in the target value of the health state of the user until a present time is output as the evaluation result.

5. The intervention content estimation apparatus according to claim 1 wherein the computer program instructions further perform to acquire, on a per user basis, most recent record information including the target value of the health state presented and the measurement value of the health state of the user after the target value of the health state is presented; and
input the most recent record information as evaluation data to the intervention content estimation model and output, as estimation data, information representing a target value of the health state to be subsequently recommended next, which is output from the intervention content estimation model in response to the input.

6. An intervention content estimation method that is executed by an information processing apparatus having a processor and a memory, the intervention content estimation method comprising:
acquiring, on a per user basis, record information that includes a target value of a health state determined based on a current health state and a future ideal health state set in advance, and a measurement value of the health state of the user after the target value of the health state is presented; and
generating, as a learning process, an intervention content estimation model by inputting the acquired record information as training data to a learning machine and causing the learning machine to perform learning such that information representing a target value of the health state to be subsequently recommended is output as an evaluation result from the learning machine.

7. The intervention content estimation method according to claim 6, further comprising:
acquiring, on a per user basis, most recent record information including the target value of the health state presented and the measurement value of the health state of the user after the target value of the health state is presented; and
inputting, as an estimation process, the acquired most recent record information as evaluation data to the intervention content estimation model and outputting, as estimation data, information representing a target value of the health state to be subsequently recommended, which is output from the intervention content estimation model in response to the input.

8. A non-transitory computer-readable medium having computer-executable instructions that, upon execution of the instructions by a processor of a computer, cause the computer to function as the intervention content estimation apparatus according to claim 1.
